(19) United States
(12) Plant Patent Application Publication
(10) Pub. No.: US 2013/0117896 P1
(43) Pub. Date: May 9, 2013

(54) Blackberry plant named 'Drisblackthree'

(50) ;

(71) Applicants: Gavin R. Sills, Gilroy, CA (US); Andrea M. Pabon, Gilroy, CA (US); Carlos D. Fear, West Malling (GB)

(72) Inventors: Gavin R. Sills, Gilroy, CA (US); Andrea M. Pabon, Gilroy, CA (US); Carlos D. Fear, West Malling (GB)

(73) Assignee: DRISCOLL STRAWBERRY ASSOCIATES, INC., Watsonville, CA (US)

(21) Appl. No.: 13/373,126

(22) Filed: Nov. 4, 2011

(51) Int. Cl. A01H 5/00

(52) U.S. Cl. PLT/203

(57) ABSTRACT

This invention relates to a new and distinct variety of Blackberry plant named ‘DrisBlackThree’, which is primarily characterized by medium-sized, oblong fruit that matures to a black color, and having an early time of bud burst, is disclosed.

### GENUS AND SPECIES

Rubus L. subgenus Rubus

### VARIETY DENOMINATION

‘DrisBlackThree’

### BACKGROUND OF THE NEW PLANT

The present invention relates to a new and distinct Blackberry cultivar designated ‘DrisBlackThree’ and botanically known as Rubus L. subgenus Rubus. This new Blackberry variety was identified in Santa Cruz County, California in July 2003 and originated from a cross between the female parent Blackberry plant ‘Driscoll Carmel’ (U.S. Plant Pat. No. 15,058) and the male parent Blackberry plant ‘Zorro’ (U.S. Plant Pat. No. 13,759). The original seedling of the new cultivar was first asexually propagated at a nursery in Santa Cruz County, California in 2003. ‘DrisBlackThree’ was subsequently asexually propagated and underwent further testing at a nursery in Santa Cruz County, California from 2003 to 2011. The present invention has been found to be stable and reproduce true to type through successive asexual propagations.

Plant Breeder's Rights for this variety have not been applied for. ‘DrisBlackThree’ has not been made publicly available or sold more than one year prior to the filing date of this application.

### SUMMARY OF THE INVENTION

The following are the most outstanding and distinguishing characteristics of this new cultivar when grown under normal horticultural practices in Santa Cruz, Calif.

1. Early bud burst; and

2. Oblong, medium-sized fruit.

### DESCRIPTION OF THE PHOTOGRAPHS

This new Blackberry plant is illustrated by the accompanying photographs, which show the fruit of the plant as well as the cane. The colors shown are as true as can be reasonably obtained by conventional photographic procedures. The photographs are of plants that are 2 years old.

FIG. 1 shows the upper and lower side of the leaves.

FIG. 2 shows the flowers, and the fruit at various stages.

FIG. 3 shows a section of a young cane.

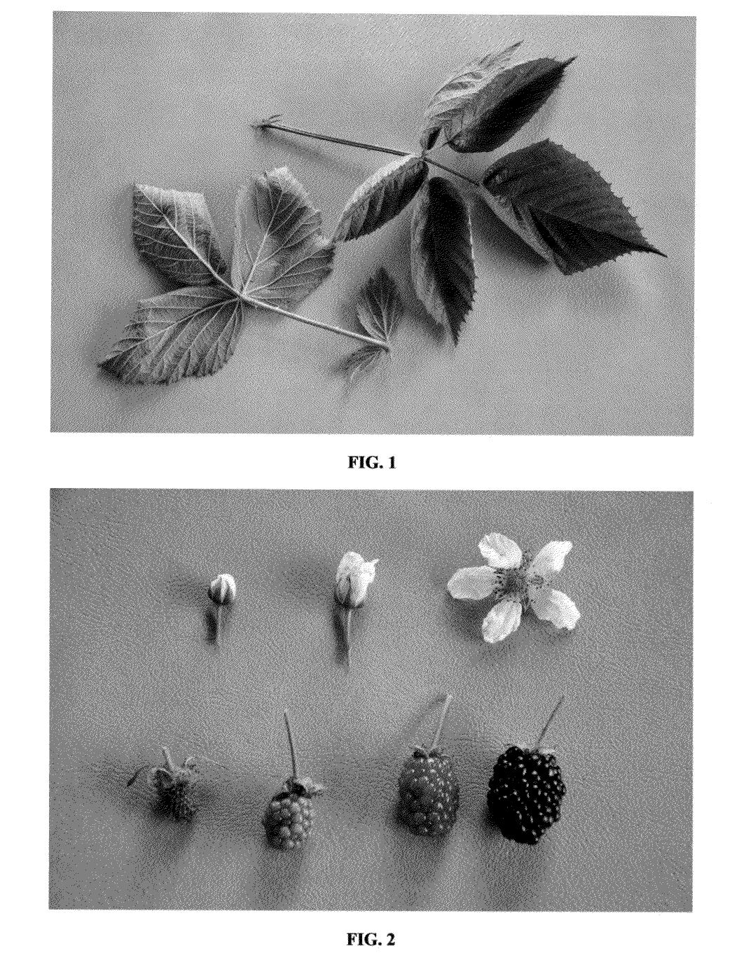

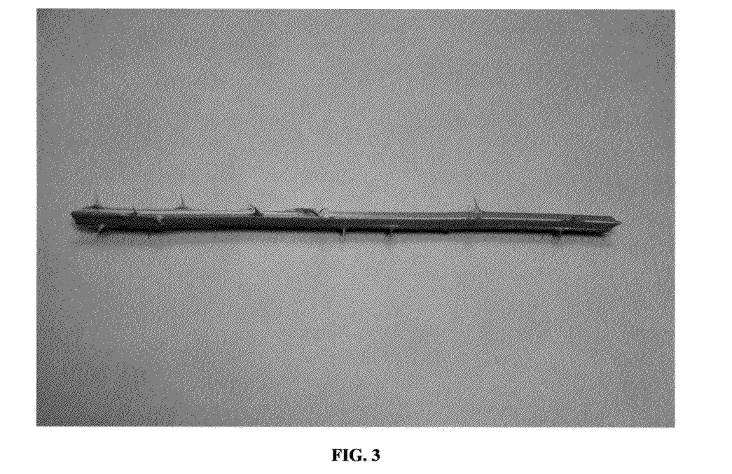

### DESCRIPTION OF THE NEW VARIETY

The following description of ‘DrisBlackThree’ is based on observations of plants that were 2 years old in Santa Cruz County, California. This description is in accordance with UPOV terminology. Color designations, color descriptions, and other phenotypical descriptions may deviate from the stated values and descriptions depending upon variation in environmental, seasonal, climatic and cultural conditions. ‘DrisBlackThree’ has not been observed under all possible environmental conditions. Color terminology follows The Royal Horticultural Society Colour Chart, London (R.H.S.) (2007 edition). Descriptive terminology is from the Plant Identification Terminology, An Illustrated Glossary 2^{nd} edition, by James G. Harris and Melinda Woolf Harris, unless where otherwise defined.

### DETAILED BOTANICAL DESCRIPTION

Classification:
   Family.—Rosaceae.
   Botanical.—Rubus L. subgenus Rubus.
   Common name.—Blackberry.
   Variety name.—‘DrisBlackThree’.
Parentage:
   Female parent.—Proprietary blackberry plant ‘Driscoll Carmel’ (U.S. Plant Pat. No. 15,058).
   Male parent.—Proprietary blackberry plant ‘Zorro’ (U.S. Plant Pat. No. 13,759).
Plant:
   Vigor (health and hardiness of plant).—Medium.
   Growth habit.—Upright to semi-upright.
   Productivity.—Medium.
   Propagation.—Tissue culture and root cuttings.
   Self-fruitfulness.—Self-fruitful.
   Time of bud burst.—Early.
Canes:
   Fruiting lateral length (4^{th} lateral from tip).—Average: Medium; 7.2 cm Range: 3.5 cm to 10.6 cm.
   Number of fruit per lateral.—Average: 6.2 Range: 4 to 9.
   Fruiting on current year's cane.—Absent.
   Cane internode length at central ⅓ of cane.—Average: 3.5 cm Range: 1.7 cm to 7.4 cm.
   Number of new canes during flowering.—Medium.
   New cane strength (observed full-grown shoot after picking).—Medium.
   Glaucosity observed on full-grown shoot after picking.—Weak.
   Spines.—Size: Medium Color: RHS N144D (Medium yellow-green) Density on central third of cane: Medium Texture: Medium Attitude of apex in relation to cane: Downwards.
   Young shoots.—Emergence from soil: Early Anthocyanin coloration (during rapid growth): Medium; RHS 144A (Medium yellow-green) Intensity of green color: Medium Number of glandular hairs: Absent or few.
   Dormant cane.—Length: Medium Diameter (in central third): Medium Cross section (from mid cane observed at end of first growing season): Angular Color: RHS 146B (Medium yellow-green) Number of branches: Medium Distribution of branches: Over whole length Anthocyanin coloration: Medium; RHS 146B (Medium yellow-green) Spines: Present Number of spines: Medium.
Leaves:
   Type.—Palmate.
   Predominant number of leaflets.—3.
   Overlapping or relative position of lateral leaflets.—Overlapping.
   Color.—Upper surface: RHS 147A (Dark yellow-green) Lower surface: RHS 146A (Medium yellow-green).
   Arrangement.—Compound (3 or more leaves per node).
   Glossiness.—Weak.
   Cross section profile.—Concave (margins rolled inwards).
   Time of leaf bud burst.—Early.
   Leaflets.—Shape: Ovate Margin: Doubly serrate Depth of incisions: Medium Apex: Truncate Base: Obtuse Rachis length between terminal leaflet and adjacent lateral leaflet: Average: 1.9 mm Range: 1.1 mm to 2.4 mm.
   Lateral leaflet (basal pair).—Length: Average: 8.4 mm Range: 6.7 mm to 10.5 mm Width: Average: 5.3 mm Range: 4.1 mm to 7.4 mm Length/width ratio: 1.6.
   Terminal leaflet.—Length: Average: 9.8 mm Range: 7.7 mm to 12.4 mm Width: Average: 6.4 mm Range: 5.3 mm to 8.6 mm Length/width ratio: 1.5 Lobing: Absent Undulation of margin (rippling of margin): Medium Blistering between veins (rugosity): Medium Shape: Ovate Tip: Truncate Base: Obtuse Margin: Doubly serrate Cross-section: U-shaped.
   Petiole.—Length average: 6.4 mm Length range: 4.3 mm to 9.5 mm Width average: 1.5 mm Width range: 1.2 mm to 1.8 mm Pigmentation: Upper surface: Heavy; RHS 147A (Dark yellow-green) Lower surface: Medium; RHS 146A (Medium yellow-green).
   Stipule.—Size: Medium Orientation: Erect.
Inflorescence:
   Time of beginning of flowering on previous year's cane.—Early.
   Flower size.—Medium.
   Flower diameter.—Average: 4.33 mm Range: 3.8 mm to 5.0 mm.
   Flower number (at 3^{rd} node from tip of lateral).—Average: 5 Range: 4 to 8.
   Petal.—Length: Average: 2.34 mm Range: 2.1 mm to 2.5 mm Width: Average: 1.07 mm Range: 1.0 mm to 1.20 mm Color: RHS 155D (White).
   Pedicel.—Length: Average: 3.69 mm Range: 2.30 mm to 5.60 mm Diameter: Average: 1.32 mm Range: 1.10 mm to 1.70 mm.
Fruit:
   Time of ripening on previous year's cane.—Early.
   Color.—Immature: RHS 151A (Medium yellow-green) Maturing: RHS 183A (Dark greyed-purple) Mature: RHS 202A (Black).
   Glossiness.—Medium.
   Shape in longitudinal section.—Oblong.
   Size.—Medium.
   Length.—Average: 27.0 mm Range: 23.0 mm to 33.0 mm.
   Width.—Average: 19.9 mm Range: 17.0 mm to 23.0 mm.
   Ratio of length to width.—1.4; Medium.
   Weight (g/fruit).—0.68.
   Soluble solids (%) (in Brix).—11.2.
   Titratable acidity.—(% as citric acid): 1.1.
   Seed weight (g/seed).—0.00287587.
   Number of druplets/fruit.—110; Medium.
   Size of druplet.—Medium.
   Firmness.—Medium.
   Yield.—Medium.
Disease, pest, and stress resistance:
   Botrytis fruit rot.—Susceptible.
   Verticillium wilt.—Susceptible.
   Leaf spots (Septoria rubi).—Moderately susceptible.
   Tetranychus urticae.—Susceptible.

### COMPARISON WITH PARENTAL AND COMMERCIAL VARIETIES

‘DrisBlackThree’ differs from the female parent ‘Driscoll Carmel’ (U.S. Plant Pat. No. 15,058) in that ‘DrisBlackThree’ matures earlier, has smaller, firmer and less sweet fruit, and lower yield.

‘DrisBlackThree’ differs from the proprietary male parent ‘Zorro’ (U.S. Plant Pat. No. 13,759) in that the fruit of ‘DrisBlackThree’ is larger, more firm and sweeter than ‘Zorro’.

‘DrisBlackThree’ differs from the commercial variety ‘Driscoll Eureka’ (U.S. Plant Pat. No. 14,765) in that ‘DrisBlackThree’ has an upright to semi-upright growth habit, early flowering, medium glossiness and oblong shaped fruit, while ‘Driscoll Eureka’ has an upright growth habit, very early flowering, strong glossiness and ovate shaped fruit.

We claim:

1. A new and distinct cultivar of Blackberry plant as described and shown herein.
